(19) United States
(12) Patent Application Publication
(10) Pub. No.: US 2010/0130866 A1
(43) Pub. Date: May 27, 2010

(54) Method for determining flow and flow volume through a vessel

(76) Inventors: Joan Carol Main, Scottsdale, AZ (US); Jamil Tajik, Fountain Hills, AZ (US); Bijoy Khandheria, Fountain Hills, AZ (US); Joan L. Lusk, Desert Hills, AZ (US); Matt Umland, Anthem, AZ (US)

Correspondence Address: QUARLES & BRADY LLP, 411 E. WISCONSIN AVENUE, SUITE 2040, MILWAUKEE, WI, 53202-4497, US

(21) Appl. No.: 12/460,224

(22) Filed: Jul. 15, 2009

Related U.S. Application Data
(60) Provisional application No. 61/135,043, filed on Jul. 16, 2008.

(51) Int. Cl. A61B 8/06

(52) U.S. Cl. 600/455

(57) ABSTRACT

A method for measuring and displaying flow and flow volume in the vessel of a subject. The method includes acquiring ultrasound data from a subject and producing a color Doppler m-mode image depicting the vessel. A 3D representation of the color Doppler m-mode image is then generated to enable an operator to identify the blood vessel and window the ultrasound data accordingly to a selected range of heart beats. Blood vessel walls are automatically identified from the windowed ultrasound data and blood flow through the vessel lumen is measured using pulsed Doppler ultrasound, which is gated to substantially exclude data from outside the lumen. Volume flow through the vessel is determined by multiplying the measured blood flow with a calculated cross-sectional area of the vessel and an image indicative of the volume is then generated and displayed.

### CROSS REFERENCE TO RELATED APPLICATIONS

This application is based on, claims the benefit of, and incorporates herein by reference U.S. Provisional Application Ser. No. 61/135,043, filed Jul. 16, 2008, and entitled “Method for Displaying Flow and Flow Volume Through a Vessel.”

### BACKGROUND OF THE INVENTION

The present invention relates to systems and methods for ultrasound imaging and, more particularly, to a system and method for using ultrasound data to measure and display flow and flow volume through a vessel with increased accuracy.

There are a number of modes in which ultrasound can be used to produce images of objects. The ultrasound transmitter may be placed on one side of the object and the sound transmitted through the object to the ultrasound receiver placed on the other side (“transmission mode”). With transmission mode methods, an image may be produced in which the brightness of each pixel is a function of the amplitude of the ultrasound that reaches the receiver (“attenuation” mode), or the brightness of each pixel is a function of the time required for the sound to reach the receiver (“time-of-flight” or “speed of sound” mode). In the alternative, the receiver may be positioned on the same side of the object as the transmitter and an image may be produced in which the brightness of each pixel is a function of the amplitude or time-of-flight of the ultrasound reflected from the object back to the receiver (“refraction”, “backscatter” or “echo” mode). The present invention relates primarily to a backscatter method for producing ultrasound images.

There are a number of well known backscatter methods for acquiring ultrasound data. In the so-called “A-mode” scan method, an ultrasound pulse is directed into the object by the transducer and the amplitude of the reflected sound is recorded over a period of time. The amplitude of the echo signal is proportional to the scattering strength of the refractors in the object and the time delay is proportional to the range of the refractors from the transducer.

In the so-called “B-mode” scan method, the transducer transmits a series of ultrasonic pulses as it is scanned across the object along a single axis of motion. The resulting echo signals are recorded as with the A-mode scan method and their amplitude is used to modulate the brightness of pixels on a display. The location of the transducer and the time delay of the received echo signals locates the pixels to be illuminated. With the B-mode scan method, enough data are acquired from which a two-dimensional image of the refractors can be reconstructed. Rather than physically moving the transducer over the subject to perform a scan it is more common to employ an array of transducer elements and electronically move an ultrasonic beam over a region in the subject.

The “M-mode” scan method is also known by its full name, “motion mode.” An M-mode scan captures returning echoes signals in only one line of a B-mode image but displays them over a time axis. Movement of structures positioned in that line can then be visualized over time. Often M-mode and B-mode are displayed together on the ultrasound monitor.

In addition, the latest ultrasound systems can now employ 3-D real-time imaging in echocardiograms. Using pulsed or continuous wave Doppler ultrasound, an echocardiogram can also produce accurate assessment of the velocity of blood or tissue at any chosen point. Doppler systems employ an ultrasonic beam to measure the velocity of moving reflectors, such as flowing blood cells or tissue. Blood velocity or tissue velocity is detected by measuring the Doppler shifts in frequency imparted to ultrasound by reflection from moving blood cells or tissue. Accuracy in detecting the Doppler shift at a particular point depends on defining a small sample volume at the required location and then processing the echoes to extract the Doppler shifted frequencies.

A Doppler system is incorporated in a real time scanning imaging system. The system provides electronic steering and focusing of a single acoustic beam and enables small volumes to be illuminated anywhere in the field of view of the instrument, whose locations can be visually identified on a two-dimensional B-mode image. A Fourier transform processor faithfully computes the Doppler spectrum backscattered from the sampled volumes, and by averaging the spectral components the mean frequency shift can be obtained. Typically the calculated velocity is used to color code pixels in the B-mode image.

Vessel flow measurements are clinically useful in the study of cerebrovascular disease, cardiovascular disease, and other clinical conditions. The use of ultrasound technology is well-known in the art as a non-invasive method to measure and image blood and other bodily fluid flow within the vessels of a living subject. For example, color spectral Doppler ultrasound imaging (SDI) can be used to determine both cross-sectional area and flow velocity within a vessel, and volume flow can be calculated as the product of flow velocity and cross sectional area. Another method, Color Velocity Imaging Quantification (CVI-Q), uses time-domain cross correlation of color B-mode ultrasound data to calculate flow volume. Flow volume can also be calculated using mean vessel velocity obtained from Doppler ultrasound data sampling and the cross sectional area measured by static B-mode ultrasound.

Both the CVI-Q and SDI techniques are inconsistent in estimations of blood flow volume, although the inconsistency is less for CVI-Q than for SDI. The error of SDI mainly comes from inaccurate diameter measurements on a static gray scale image, with the assumption of a stable vessel. Small errors in the diameter measurement result in large errors in the calculation of cross-sectional area and, thus, flow volume. Because the physiological anatomic diameter in systole or diastole varies and may differ by as much as 10%, the diameter variation alone can account for flow volume errors of up to 20%. Although CVI-Q provides more accurate diameter measurements than SDI, both techniques are subject to other significant sources of error, including angle correction error, turbulent flow error, off axis sampling error, and error caused by the pulsing of the vessel being measured.

Therefore, it would be desirable to have a system and method for measuring and displaying flow and flow volume through a vessel that provides a more accurate measurement of cross sectional area and beat to beat variation than is provided by the present methods. Furthermore, it would be desirable that such a system and method could be utilized to perform such an analysis non-invasively.

### SUMMARY OF THE INVENTION

The present invention overcomes the aforementioned drawbacks by providing a method to non-invasively and more accurately measure flow and flow volume in the vessels of patients. The method includes comprises the steps of acquiring ultrasound data from the subject and producing a 2D Doppler image, generating a 3D representation of the 2D Doppler image in which blood vessel walls are identified by changes in at least one of color, hue, brightness, and intensity, windowing the acquired ultrasound data based on the 3D representation, and automatically identifying a selected depth range in the windowed ultrasound data stretching from a first identified wall of a blood vessel to a second identified wall of the blood vessel. The method further comprises the steps of calculating a cross-sectional area of the blood vessel using the selected depth range in the windowed ultrasound data, performing a pulsed Doppler ultrasound scan to acquire pulsed Doppler ultrasound data, measuring blood flow velocity in the subject within the selected depth range using pulsed Doppler ultrasound data, determining a volume flow through the blood vessel from the measured blood velocity and calculated cross-sectional area, and generating an image indicative of volume flow through the blood vessel from the determined volume flow.

The invention is not limited to these aspects, and various other features of the present invention will be made apparent from the following detailed description and the drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram of an ultrasonic imaging system which employs the present invention;

FIG. 2 is a block diagram of a transmitter which forms part of the system of FIG. 1;

FIG. 3 is a block diagram of a receiver which forms part of the system of FIG. 1;

FIG. 4 is a flow chart setting forth the steps of a method for non-invasively calculating and displaying flow and flow volume through a vessel in accordance with the present invention using the system of FIGS. 1-3;

FIG. 5 is a color-coded, two-dimensional linear display of M-mode velocities within a vessel plotted against time;

FIG. 6 is a color-coded, three dimensional display showing the derived flow profile of each of the pixel values along the M-mode line plotted against time, derived from the two dimensional display of FIG. 5;

FIG. 7 is a color-coded, three dimensional display showing filtered M-mode data that can be used to check the accuracy of the cross sectional area calculations derived from FIG. 6;

FIG. 8 is a discontinuous, color-coded, three dimensional integrated waterfall display of volume flow through a vessel showing the individual subsets of the full velocity profile;

FIG. 9 is a display showing graphical heartbeat information; and

FIG. 10 is a color coded two dimensional display showing average mean volume flow superimposed over graphical heartbeat information.

### DETAILED DESCRIPTION OF THE INVENTION

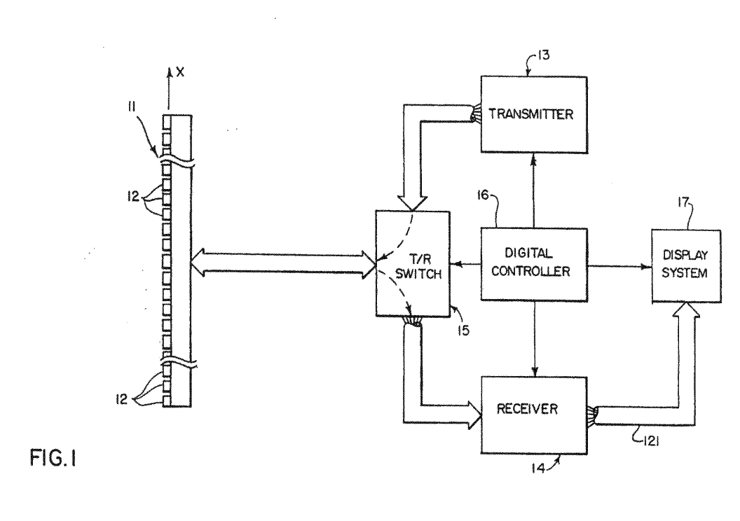

Referring particularly to FIG. 1, an ultrasonic imaging system includes a transducer array 11 comprised of a plurality of separately driven elements 12 that each produce a burst of ultrasonic energy when energized by a pulse produced by a transmitter 13. The ultrasonic energy reflected back to the transducer array 11 from the subject under study is converted to an electrical signal by each transducer element 12 and applied separately to a receiver 14 through a set of switches 15. The transmitter 13, receiver 14 and the switches 15 are operated under the control of a digital controller 16 responsive to the commands input by the human operator. A complete scan is performed by acquiring a series of echoes in which the switches 15 are set to their transmit position, the transmitter 13 is gated on momentarily to energize each transducer element 12, the switches 15 are then set to their receive position, and the subsequent echo signals produced by each transducer element 12 are applied to the receiver 14. The separate echo signals from each transducer element 12 are combined in the receiver 14 to produce a single echo signal that is employed to produce a line in an image on a display system 17.

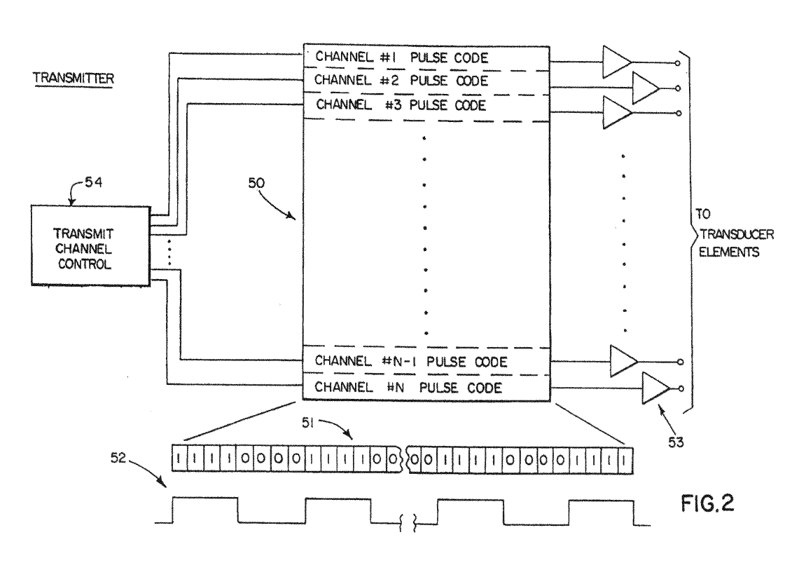

Referring particularly to FIG. 2, the transmitter 13 includes a set of channel pulse code memories which are indicated collectively at 50. Each pulse code memory 50 stores a bit pattern 51 that determines the frequency of the ultrasonic pulse 52 that is to be produced. This bit pattern is read out of each pulse code memory 50 by a master clock and applied to a driver 53 which amplifies the signal to a power level suitable for driving the transducer 11. In the example shown in FIG. 2, the bit pattern is a sequence of four “1” bits alternated with four “0” bits to produce a 5 MHz ultrasonic pulse 52. The transducer elements 11 to which these ultrasonic pulses 52 are applied respond by producing ultrasonic energy.

As indicated above, to steer the transmitted beam of the ultrasonic energy in the desired manner, the pulses 52 for each of the N channels must be produced and delayed by the proper amount. These delays are provided by a transmit control 54 which receives control signals from the digital controller 16 (FIG. 1). When the control signal is received, the transmit control 54 gates a clock signal through to the first transmit channel 50. At each successive delay time interval thereafter, the clock signal is gated through to the next channel pulse code memory 50 until all the channels to be energized are producing their ultrasonic pulses 52. Each transmit channel 50 is reset after its entire bit pattern 51 has been transmitted and the transmitter 13 then waits for the next control signal from the digital controller 16.

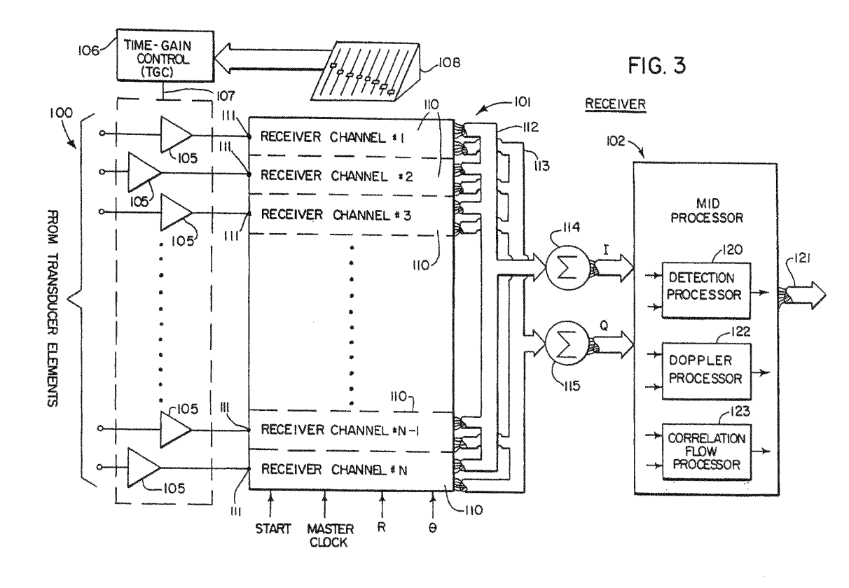

Referring particularly to FIG. 3, the receiver 14 is comprised of three sections: a time-gain control section 100, a beam forming section 101, and a mid processor 102. The time-gain control section 100 includes an amplifier 105 for each of the N receiver channels and a time-gain control circuit 106. The input of each amplifier 105 is connected to a respective one of the transducer elements 12 to receive and amplify the echo signal that it receives. The amount of amplification provided by the amplifiers 105 is controlled through a control line 107 that is driven by the time-gain control circuit 106. As is well known in the art, as the range of the echo signal increases, its amplitude is diminished. As a result, unless the echo signal emanating from more distant reflectors is amplified more than the echo signal from nearby reflectors, the brightness of the image diminishes rapidly as a function of range (R). This amplification is controlled by the operator who manually sets TGC linear potentiometers 108 to values which provide a relatively uniform brightness over the entire range of the scan. The time interval over which the echo signal is acquired determines the range from which it emanates, and this time interval is divided into segments by the TGC control circuit 106. The settings of the potentiometers are employed to set the gain of the amplifiers 105 during each of the respective time intervals so that the echo signal is amplified in ever increasing amounts over the acquisition time interval.

The beam forming section 101 of the receiver 14 includes N separate receiver channels 110. Each receiver channel 110 receives the analog echo signal from one of the TGC amplifiers 105 at an input 111, and it produces a stream of digitized output values on an I bus 112 and a Q bus 113. Each of these I and Q values represents a sample of the echo signal envelope at a specific range (R). These samples have been delayed in the manner described above such that when they are summed at summing points 114 and 115 with the I and Q samples from each of the other receiver channels 110, they indicate the magnitude and phase of the echo signal reflected from a point P located at range R on the ultrasonic beam.

Referring still to FIG. 3, the mid processor section 102 receives the beam samples from the summing points 114 and 115. The I and Q values of each beam sample is a digital number that represents the in-phase and quadrature components of the magnitude of the reflected sound from a point P. The mid processor 102 can perform a variety of calculations on these beam samples, where choice is determined by the type of image to be reconstructed. For example, if a conventional magnitude image is to be produced, a detection process indicated at 120 is implemented in which a digital magnitude M is calculated from each beam sample and output at 121.

M=\sqrt{I²+Q²}

The detection process 120 may also implement correction methods such as that disclosed in U.S. Pat. No. 4,835,689. Such correction methods examine the received beam samples and calculate corrective values that can be used in subsequent measurements by the transmitter 13 and receiver 14 to improve beam focusing and steering. Such corrections are necessary, for example, to account for the non-homogeneity of the media through which the sound from each transducer element travels during a scan.

The mid processor may also include a Doppler processor 122. Such Doppler processors often employ the phase information (φ) contained in each beam sample to determine the velocity of reflecting objects along the direction of the beam (i.e. direction from the transducer 11), where: φ=tan⁻¹(I/Q).

The mid processor may also include a correlation flow processor 123, such as that described in U.S. Pat. No. 4,587,973, issued May 13, 1986 and entitled “Ultrasonic Method Can Means For Measuring Blood Flow And The Like Using Autocorrelation”. Such methods measure the motion of reflectors by following the shift in their position between successive ultrasonic pulse measurements.

As will be described in detail below, the present invention utilizes the above-described systems to accurately analyze and calculate the cross sectional area of a vessel wall, measure the flow velocity through the vessel, calculate the flow volume through the vessel, and produce color coded displays of flow velocity and flow volume.

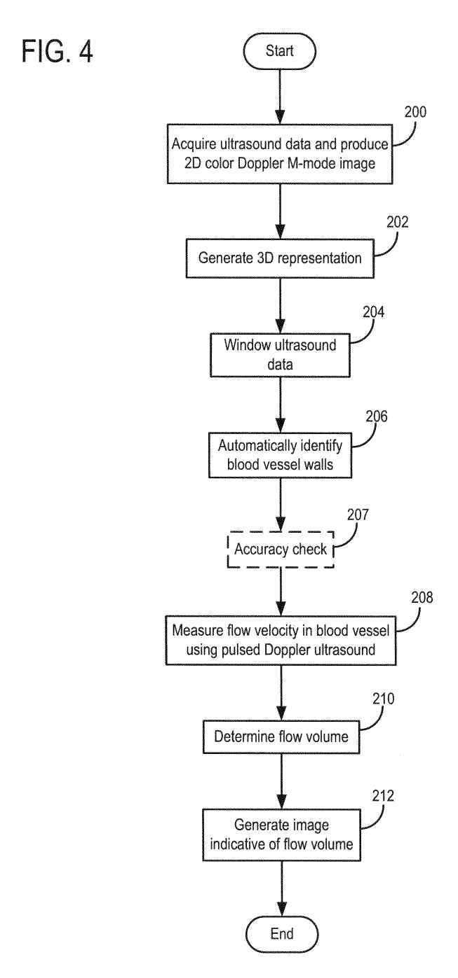

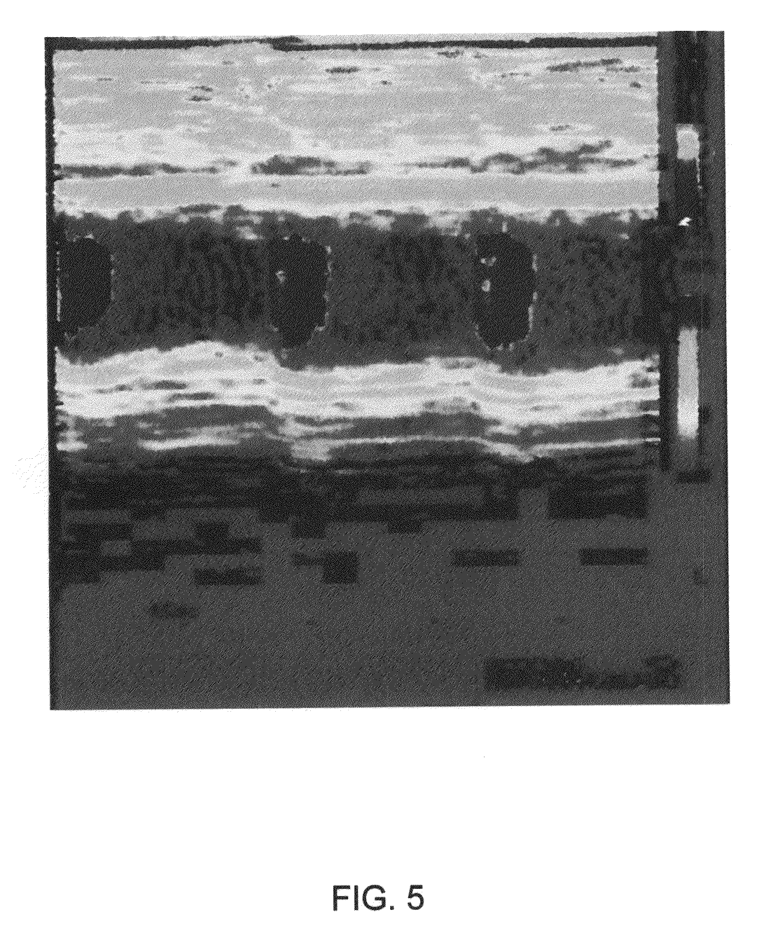

Referring now to FIGS. 4 and 5, a method in accordance with the present invention and using an ultrasound system such as that disclosed in FIGS. 1-3 begins at process block 200 with the acquisition of ultrasound data from a region-of-interest in a subject and the production of a 2D color Doppler M-mode ultrasound image showing flow through the imaged vessel. In color Doppler M-mode, information is acquired continuously along the scan line, color encoded, and displayed in a 2-D time-sequence of lines. This can be achieved by transmitting an ultrasonic pulse into the subject and receiving echoes along a given scan line. The echo signal amplitudes can be detected and displayed as a grayscale underlying anatomical display. A series of additional pulses along the scan line provide data that are Doppler processed can be used measure the velocity of motion at each point along the scan line. For example, FIG. 5 shows a 2D color Doppler M-mode image that includes an underlying grayscale image in which the vertical direction corresponds to a distance from the ultrasound transducer along a given scan line, the horizontal direction corresponds to a temporal axis, and the intensity of pixels corresponds to acoustic impedance. Color Doppler data is superimposed on the underlying grayscale to show changes in velocity at each point along the scan line over time. Thus, the color of the image will vary with the direction and intensity of the motion of fluid through the vessel. In this case, Blue indicates motion away from the transducer while red indicates motion towards the transducer. It should be noted that the single lines of color Doppler data can be used to calculate the mean velocites of fluid flow through the vessel and that it is beneficial to continuously save and display the original acquired ultrasound data.

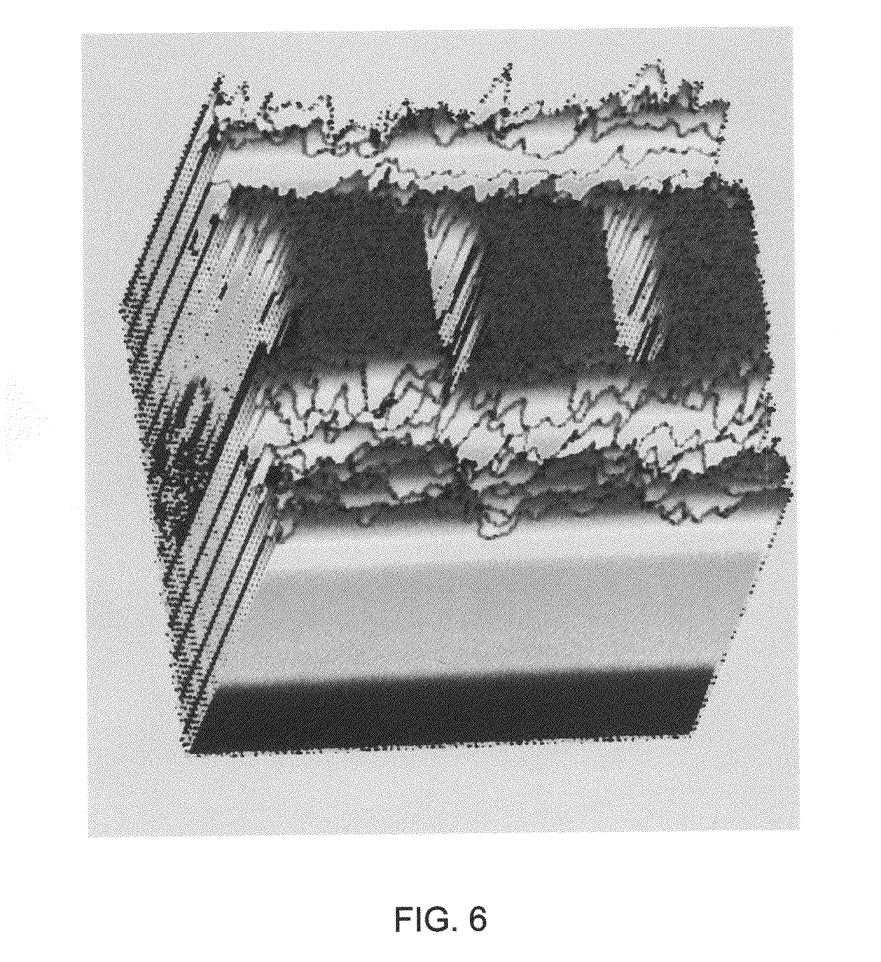

Referring particularly to FIGS. 4 and 6, at process block 202, a 3D color representation of the 2D color Doppler M-mode image is generated. This can include is analyzing the ultrasound image data and display the derived flow profile of each of the pixel values along the M-mode line. Flow profiles can include such subsets of the full velocity profile as average mean velocity at each line, peak mean velocity at each line, and the modal of velocities within the display and each color in the 3D display can represent a different subset of the full velocity profile. The 3D color representation is used in the present invention to precisely and accurately identify the locations of the vessel walls. Tissue boundaries such as vessel walls can be precisely identified in the 3D representation by transitions in color, hue, brightness or intensity. It is contemplated that the 3D representations can be shown using a dual screen display that also shows the original 2-D M-mode image or using a quad screen or live quad screen format display. FIG. 6 shows exemplary 3D representation of the 2D color Doppler M-mode image generated using NIH-Image (NIH, Bethesda, Md.), a Macintosh-based image processing and analysis program developed at the Research Services Branch (RSB) of the National Institute of Mental Health (NIMH), part of the National Institutes of Health (NIH). A Windows-based version of Image, called Scion Image for Windows (Scion Corporation, Frederick, Md.), can also acquire, display, edit, enhance, analyze and animate images, and supports the conversion of 2-D images into 3-D displays.

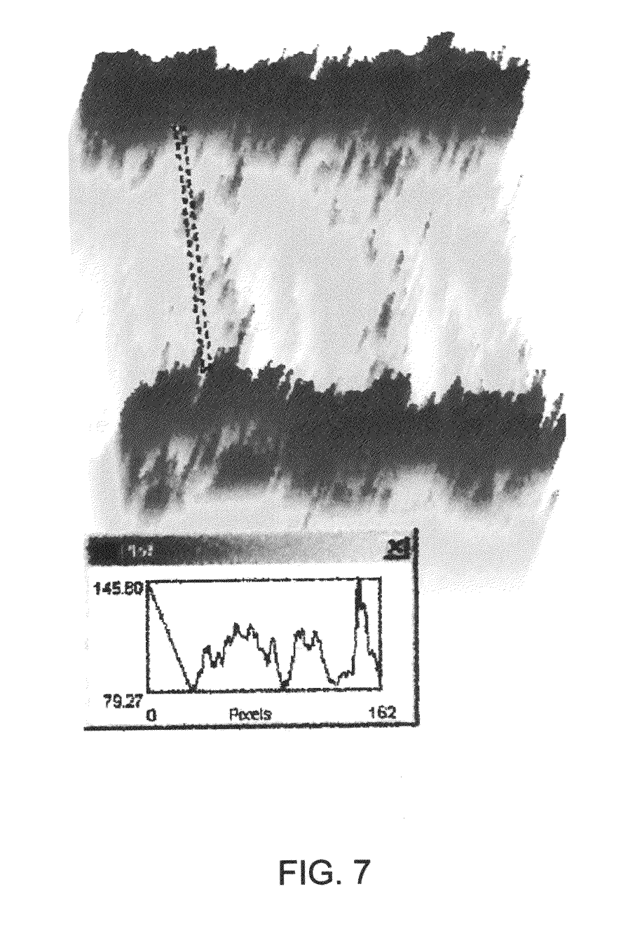

Referring to FIGS. 4 and 7, at process block 204, the ultrasound data is windowed based on the 3D representation of the 2D color Doppler M-mode image. This can be performed manually by visually inspection of the 3D representation and the exclusion of data outside a selected range of heart beats from further analysis. Since blood vessel walls are not necessarily straight or uniform through time, it is easier for an operator to accurately identify blood vessel walls and window the ultrasound data appropriately using the 3D representation, rather than the 2D color Doppler M-mode image. At process block 206, blood vessel walls are automatically identified in the windowed ultrasound data. Precise identification of blood vessel walls can be performed using Image or similar image processing software. As indicated at process block 207, the automatically identified blood vessel boundaries can optionally be checked for accuracy via manual visual inspection. A software filter can be applied to the color display data to minimize user variability in vessel wall identification. FIG. 7 is an example of a filtered 3-D image that can be used to check the accuracy of wall identification and diameter measurements. The lumen of the vessel is coded yellow and the vessel walls are indicated by orange coloring. The vessel diameter, indicated in FIG. 7 by the dotted line, can be checked visually against the color contrasts of the filtered image.

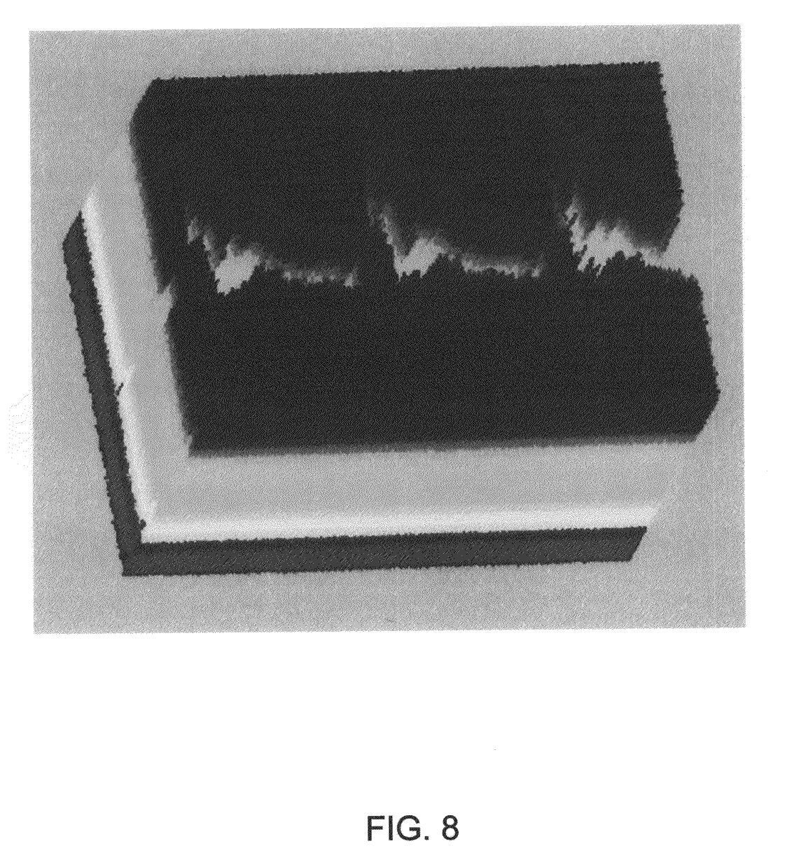

Referring to FIGS. 4 and 8, once the vessel walls are precisely identified, the velocity of blood flow within the vessel can be measured using pulsed Doppler ultrasound at process block 208. This includes the acquisition of pulse Doppler ultrasound data from a selected depth range spanning between the vessel walls identified at process block 206. In pulsed Doppler ultrasound, the reception of echo signals is gated so that velocity measurements are only taken substantially within the selected range. This allows a more accurate determination of blood velocity, as it screens potentially error-inducing signal from outside the blood vessel. At process block 210, volume flow through the vessel is determined, for example, by multiplying the mean blood velocity with the cross-sectional area of the blood vessel. The distance between the blood vessel walls identified in process block 206 can be taken as the diameter of the blood vessel and the cross-sectional area can thus be calculated as π(diameter)². At process block 212, volume flow data can be displayed in a number of different ways. One option is to show volume flow information as a discontinuous color-coded 3-D waterfall display as depicted in FIG. 8. Other display options may include additional processing of the ultrasound data. For example, an algorithm using a flow profile derived from the original 2-D color Doppler M-mode data can be used to quantify and display volume flow. The algorithm calculates the integral of the velocities over time. Each color in FIG. 8 codes a different set of volume calculations. The colors together provide a complete volume flow profile.

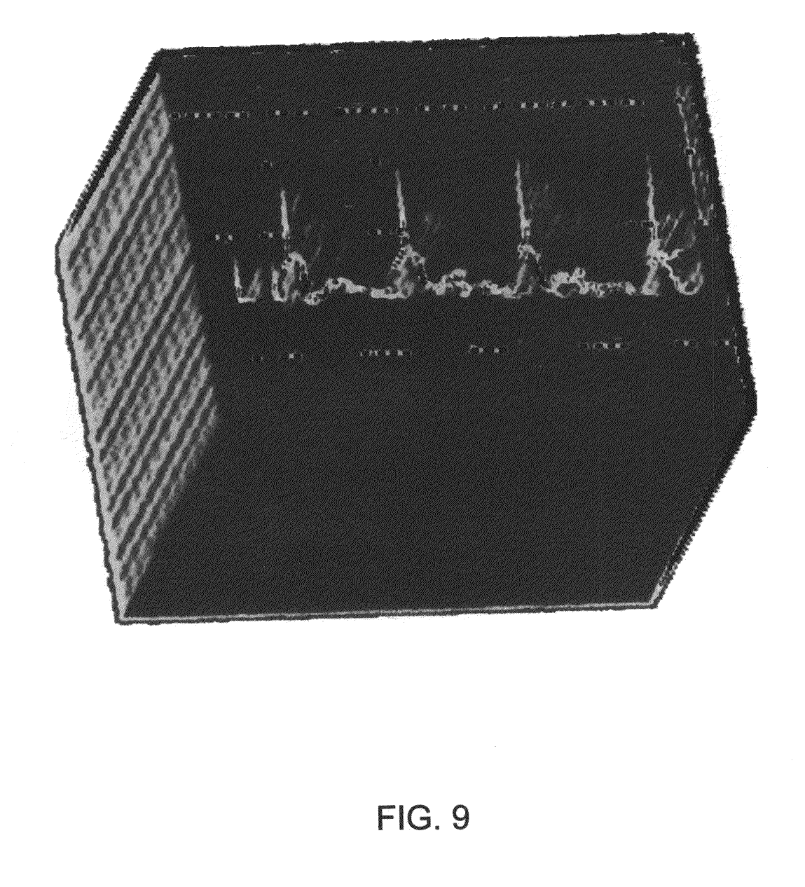

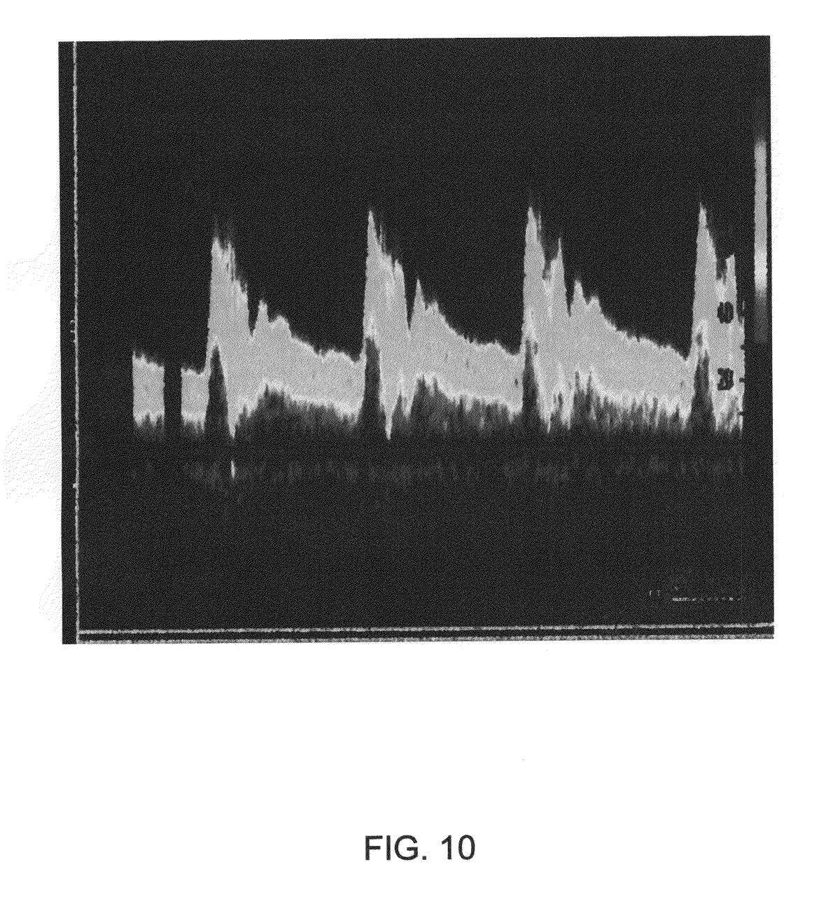

Referring to FIGS. 9 and 10, volume flow data can also be displayed as a modal display showing the average mean volume superimposed over graphical heartbeat information. FIG. 9 includes graphical heartbeat information that can be used in addition to cross sectional area and tissue velocity imaging to create a richer volume flow profile. In FIG. 10, the modal flow volume is superimposed on a graphical heartbeat information image and is shown as a band of green extending from left to right across the display.

The present invention has been described in terms of a preferred embodiment, and it should be appreciated that many equivalents, alternatives, variations, and modifications, aside from those expressly stated, are possible and within the scope of the invention. Therefore, the invention should not be limited to a particular described embodiment.

1. A method of producing an ultrasound image indicative of a volume flow in a subject, the method comprising the steps of:
a) acquiring ultrasound data from the subject and producing a 2D Doppler image;
b) generating a 3D representation of the 2D Doppler image in which blood vessel walls are identified by changes in at least one of color, hue, brightness, and intensity;
c) windowing the acquired ultrasound data based on the 3D representation;
d) automatically identifying a selected depth range in the windowed ultrasound data stretching from a first identified wall of a blood vessel to a second identified wall of the blood vessel;
e) calculating a cross-sectional area of the blood vessel using the selected depth range in the windowed ultrasound data;
f) performing a pulsed Doppler ultrasound to acquire pulsed Doppler ultrasound data;
g) measuring blood flow velocity in the subject within the selected depth range using pulsed Doppler ultrasound data;
h) determining a volume flow through the blood vessel from the blood velocity measured in step d) and the cross-sectional area of the blood vessel calculated in step e); and
i) generating an image indicative of volume flow through the blood vessel from the volume flow determined in step h).

2. The method as recited in claim 2 wherein step c) includes windowing the acquired ultrasound data based on manual visual inspection of the 3D representation.

3. The method as recited in claim 2 wherein the manual visual inspection of the 3D representation includes identifying at least one of a blood vessel wall and a selected range of heart beats.

4. The method as recited in claim 3 wherein step c) includes windowing the acquired ultrasound data to be within the selected range of heart beats.

5. The method as recited in claim 1 wherein step e) includes determining the cross-sectional area of the blood vessel from a diameter of the blood vessel that is selected to be substantially equal to the selected depth range.

6. The method as recited in claim 1 further comprising step j) displaying the determined volume flow as a modal display showing an average mean flow volume superimposed over graphical heartbeat information

7. The method as recited in claim 1 wherein step g) includes gating the reception of echo signals to substantially prevent the reception of echo signals corresponding to regions outside the selected depth range.

8. The method as recited in claim 1 wherein the 2D Doppler image is a color M-mode 2D Doppler image.
